(19) United States
(12) Patent Application Publication
(10) Pub. No.: US 2024/0325289 A1
(43) Pub. Date: Oct. 3, 2024

(54) MEDICINAL MUSHROOM-INFUSED TOPICAL COMPOSITION FOR TATTOO AFTERCARE

(71) Applicant: Cynthia Gomez, Denver, CO (US)

(72) Inventor: Cynthia Gomez, Denver, CO (US)

(73) Assignee: Cynthia Gomez, Denver, CO (US)

(21) Appl. No.: 18/615,128

(22) Filed: Mar. 25, 2024

Related U.S. Application Data
(60) Provisional application No. 63/455,558, filed on Mar. 30, 2023.

(51) Int. Cl. A61K 8/9728; A61K 8/67; A61K 8/92; A61K 8/9794; A61Q 17/04; A61Q 19/00

(52) U.S. Cl. CPC A61K 8/9728; A61K 8/671; A61K 8/676; A61K 8/92; A61K 8/9794; A61Q 17/04; A61Q 19/00

(57) ABSTRACT

The present invention relates to a topical aftercare composition designed for the care and maintenance of tattooed skin. The composition features a unique blend of medicinal mushroom extracts, including Chaga, Lion's Mane, and Reishi, known for their healing properties. These extracts are combined with aloe vera gel and non-comedogenic carrier oils to create a homogenous mixture that promotes the healing of tattooed skin. An unexpected benefit of the invention is its inherent UV radiation protective qualities, derived from the high melanin content in the Chaga mushroom extract. This feature provides added protection against the fading and degradation of tattoos due to sun exposure. The composition is formulated to be hypoallergenic and non-irritating, making it suitable for all skin types. It is intended for use starting 48-72 hours after tattooing and for ongoing maintenance to keep the tattooed skin vibrant and healthy. The invention stands out in the realm of tattoo aftercare for its synergistic blend of ingredients that not only expedite the healing process but also offer long-term benefits for the preservation of tattoo quality.

### CROSS-REFERENCE TO RELATED APPLICATIONS

This application claims the benefit of and priority to U.S. Provisional Patent Application 63/455,558, filed on Mar. 30, 2023, which is hereby incorporated by reference in its entirety.

### BACKGROUND OF THE INVENTION

### 1. Field of the Invention

The present invention pertains to the field of dermatological skincare, specifically to the formulation of topical aftercare products designed for the care and maintenance of tattooed skin. It falls within the realm of cosmetic and therapeutic skin treatments that aim to enhance the healing of the epidermis following the trauma induced by tattooing procedures.

The field relates to the intersection of cosmetology, dermatology, and alternative medicine, leveraging the pharmacological properties of natural extracts to facilitate skin recovery and health. It is particularly relevant to the niche market of tattoo aftercare, which seeks to address the unique challenges associated with the healing of tattooed skin, such as the prevention of infection, reduction of inflammation, and the minimization of scarring or color fading.

The field also relates to the immune-stimulating and wound-healing properties of medicinal mushrooms. The field of the invention also encompasses the development of aftercare lotions, salves, gels, and other topical applications that incorporate bioactive compounds for improved skin healing and tattoo preservation.

### ii. Relevant Art

There remain several critical issues inherent in the current practices of tattoo aftercare. Traditional methods for tattoo aftercare have predominantly involved the use of mild soaps for cleansing and unscented moisturizers to hydrate the skin throughout the healing phases. However, these conventional products lack specific ingredients that actively contribute to the skin's natural healing processes. Moreover, the tattoo industry has commonly recommended petroleum-based products, such as Aquaphor, which have been found to be counterproductive. These products tend to over-moisturize scabs due to their slow absorption rate, potentially leading to clogged pores and a complicated healing process.

The over-moisturization of tattooed skin can create an environment that is conducive to bacterial growth, increasing the risk of infection and potentially leading to longer healing times and compromised tattoo quality. The occlusive nature of petroleum-based products can trap bacteria and other contaminants against the skin, which is particularly problematic during the critical initial healing stages when the skin is most vulnerable.

Additionally, the use of these heavy, occlusive products often results in a lack of breathability for the skin. This can delay the natural cell turnover and oxygenation processes that are essential for proper healing. As a result, the tattooed area may experience increased itchiness, irritation, and discomfort, which can tempt individuals to scratch or pick at the healing tattoo, further risking damage and infection.

Another significant issue with current aftercare products is their failure to address the long-term care of the tattoo. While initial healing is crucial, the maintenance of tattoo vibrancy and clarity over time is often overlooked. Exposure to ultraviolet (UV) radiation can cause tattoos to fade prematurely, and most aftercare products do not offer any protection against these harmful rays.

Furthermore, many individuals with sensitive skin or allergies find that the limited selection of aftercare products available cannot meet their needs. The use of synthetic fragrances, preservatives, and other potential irritants in aftercare products can lead to allergic reactions, exacerbating the challenges of healing and maintaining a tattoo.

In light of these issues, there is a clear need for an aftercare product that not only aids in the efficient and effective healing of the tattooed skin but also supports the long-term health and appearance of the tattoo. The invention seeks to fulfill this need by providing a formulation that is gentle, promotes rapid healing, and incorporates ingredients that offer sustained benefits for the skin and the integrity of the tattoo.

### SUMMARY OF THE INVENTION

The preferred embodiment presents a novel approach to tattoo aftercare by introducing a unique formulation that synergistically combines the therapeutic properties of mushroom extracts with the soothing effects of aloe vera gel and the safety of non-comedogenic carrier oils. This innovative blend not only circumvents the common issues associated with traditional aftercare products, such as pore-clogging and delayed healing, but it also enhances the body's immune response, thereby expediting the natural healing process. The selected medicinal mushroom extracts, including Chaga, Lion's Mane, and Reishi, are renowned for their wound-healing capabilities, which are attributed to their ability to stimulate immune epithelial cells, the extracellular matrix, cytokines, growth factors, reactive oxygen species (ROS), and various inflammatory intermediates.

The preferred embodiment further innovates in the realm of long-term tattoo care. It provides a product that is not only beneficial during the critical initial healing phase but also continues to support skin hydration and health long after the tattoo has healed. The high melanin content found in certain mushroom extracts, such as Chaga, offers an additional advantage by providing a natural layer of UV radiation protection. This feature is particularly valuable as it aids in maintaining the tattoo's vibrancy and preventing premature fading due to sun exposure.

The preferred embodiment of the invention represents a significant leap forward in the field of tattoo aftercare. It addresses the shortcomings of existing aftercare products by eliminating the risk of over-moisturization and clogged pores, which are often associated with petroleum-based products. Moreover, it fills the gap in the market for a product that offers long-term protection against UV radiation, a common cause of tattoo degradation. The formulation is designed to be safe for all skin types, non-toxic, and hypoallergenic, minimizing the likelihood of adverse or allergic reactions. Its application is recommended for both fresh and aging tattoos, ensuring that the skin remains vibrant, healthy, and well-protected throughout the life of the tattoo. The invention thus provides a comprehensive solution that promotes healing, preserves tattoo quality, and enhances overall skin health.

The preferred embodiment of the invention further provides a significant advancement in tattoo aftercare by solving the problems of ineffective healing ingredients in current products, the risk of over-moisturization and clogged pores associated with petroleum-based products, and the lack of long-term tattoo protection from UV rays. The result is a product that is safe, effective, and beneficial for both the initial healing phase and the ongoing maintenance of tattooed skin.

### BRIEF DESCRIPTION OF THE FIGURES

FIG. 1 depicts a graph presenting the ratios of ingredients in an exemplary gel embodiment of the invention.

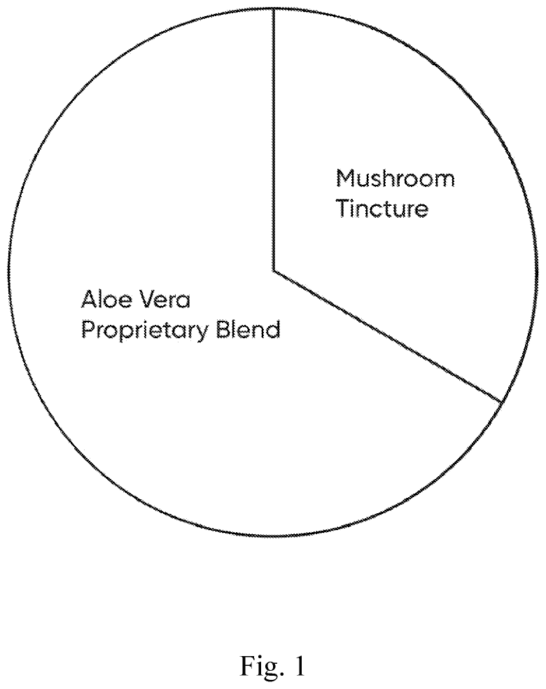

FIG. 2 depicts a graph presenting the ratios of ingredients in an exemplary salve embodiment.

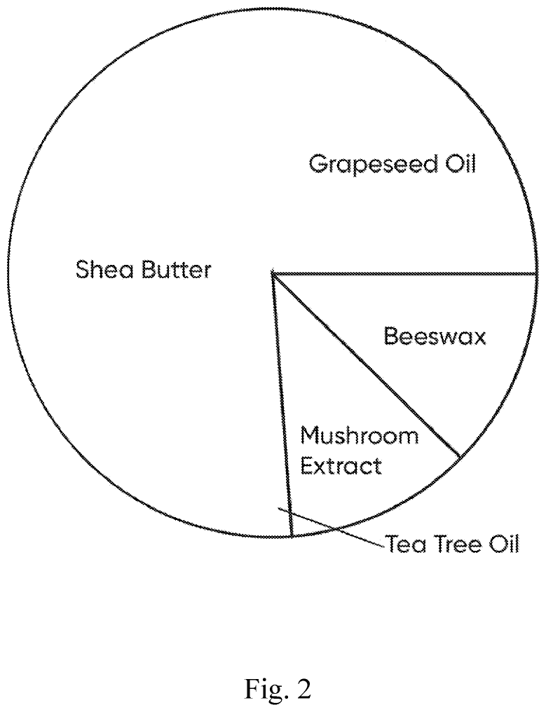

### DETAILED DESCRIPTION OF THE INVENTION

The preferred embodiment of the present invention provides a detailed formulation and method for the preparation and application of a topical aftercare product specifically designed for the care of tattooed skin. The invention is a homogenous mixture that combines the healing properties of medicinal mushroom extracts with a proprietary blend of aloe vera gel and non-comedogenic carrier oils, along with other cosmetic grade ingredients that are well-known for their skin care benefits.

### Formulation

The core of the preferred embodiment lies in its unique blend of ingredients. The primary active components are extracts from medicinal mushrooms comprising Chaga, Lion's Mane, and Reishi. These extracts are rich in beta-glucans, antioxidants, and anti-inflammatory compounds that promote healing and skin health. The extracts are obtained through a process that ensures the preservation of their medicinal properties and are then mixed in predetermined quantities with aloe vera gel, which serves as a hydrating and soothing agent.

The invention in its preferred embodiment utilizes a proprietary extraction process that maximizes the bioavailability of the active compounds found in Chaga, Lion's Mane, and Reishi mushrooms. This process involves a dual-phase extraction method that combines both hot water and ethanol extractions, ensuring a full spectrum of healing compounds is captured. The process is carefully controlled to maintain the integrity of the sensitive compounds and to prevent degradation. Additionally, the invention employs a novel emulsification technique that ensures a stable and homogenous mixture of the oil and water-based components, which is critical for maintaining the efficacy of the active ingredients throughout the product's shelf life.

The selection of Chaga, Lion's Mane, and Reishi mushroom extracts was based on extensive research into their individual and combined therapeutic properties. The preferred embodiment's formulation leverages the unique synergy between these extracts and the aloe vera gel, resulting in enhanced anti-inflammatory and antioxidant effects that are not present in other aftercare products. This synergistic combination has been found to significantly improve the healing process of tattooed skin, an effect that was neither taught nor suggested in prior art.

To enhance the product's skin-friendly properties, non-comedogenic carrier oils such as grapeseed oil and tea tree oil are incorporated. These oils are selected for their ability to moisturize without clogging pores and for their natural antiseptic properties. Additional ingredients may include beeswax, shea butter, and vitamins A and C, which provide a protective barrier, further nourishment, and antioxidant benefits.

Embodiments of the invention may also contain emollients, emulsifiers, and stabilizers to achieve the desired consistency and stability of the final product. Optional scent variations can be added using natural essential oils, provided they do not irritate the skin or detract from the product's healing properties.

An unexpected and advantageous discovery associated with embodiments of the invention was the identification of inherent UV radiation protective qualities within the final product. This serendipitous finding emerged from the high melanin content naturally present in the Chaga mushroom extract, a primary active ingredient in the formulation. Melanin is known for its ability to absorb harmful UV rays, thereby offering a layer of protection against solar radiation. When incorporated into the aftercare product, the melanin-rich Chaga extract imparts these protective properties to the skin, effectively shielding the tattooed area from the damaging effects of UV exposure.

The significance of this UV protective effect is a key aspect of the preferred embodiment, as it extends the utility of the invention beyond mere hydration and healing. Fresh tattoos, which are particularly vulnerable to sun damage during the healing phase, benefit from the added protection that helps prevent fading and preserves the integrity of the tattoo's design. Aging tattoos also gain from this feature, as the protective layer helps maintain the vibrancy of the ink over time, combating the natural degradation that occurs with prolonged UV exposure. The presence of such a property in a tattoo aftercare product is highly beneficial, distinguishing the invention from existing aftercare solutions that do not offer integrated UV protection.

### Active Components and Ratios:

The primary active components of the preferred embodiment are the extracts from medicinal mushrooms, specifically Chaga, Lion's Mane, and Reishi. These extracts are known for their high content of beta-glucans, antioxidants, and anti-inflammatory compounds. The extracts are prepared using a dual extraction process—both water and alcohol extractions—to ensure a full spectrum of the mushrooms' bioactive compounds are captured.

In an exemplary embodiment, the ratios of the active compounds are as follows:
Chaga Extract: 5-10% by weight
Lion's Mane Extract: 3-7% by weight
Reishi Extract: 3-7% by weight

These extracts are then combined with aloe vera gel, which acts as a hydrating and soothing agent. The aloe vera gel is used in a concentration that ensures the product is easily spreadable and quickly absorbed by the skin without leaving a residue.

In an exemplary embodiment, the ratios of the aloe vera gel are as follows:
Aloe Vera Gel: 40-60% by weight

### Carrier Oils and Additional Ingredients:

To the mixture of mushroom extracts and aloe vera gel, non-comedogenic carrier oils are added. These oils are selected for their moisturizing properties and compatibility with sensitive skin.

In an exemplary embodiment, the ratios of the oils are as follows:
Grapeseed Oil: 5-10% by weight
Tea Tree Oil: 0.5-2% by weight

The formulation may also include natural waxes and butters to provide a protective barrier and additional moisturizing benefits.

In an exemplary embodiment, the ratios of the natural waxes and butters are as follows:
Beeswax: 1-5% by weight
Shea Butter: 2-8% by weight

Vitamins A and C are incorporated as antioxidants to support skin health and aid in the healing process.

In an exemplary embodiment, the ratios of the Vitamins A and C are as follows:
Vitamin A (Retinyl Palmitate): 0.1-0.5% by weight
Vitamin C (Ascorbic Acid): 0.1-0.5% by weight

In an exemplary embodiment, the invention incorporates advanced delivery systems such as liposomal encapsulation, which enhances the penetration of the mushroom extracts into deeper layers of the skin. This targeted delivery system ensures that the active ingredients are released directly at the site of the tattooed tissue, providing localized benefits and reducing the potential for systemic absorption. The controlled-release mechanism also maintains a consistent therapeutic effect over an extended period, which is crucial for the healing process.

### Emollients, Emulsifiers, and Stabilizers:

To achieve the desired consistency and ensure the stability of the emulsion, the following ingredients may be included, in the following ratios in an exemplary embodiment:
Emollient (e.g., Glycerin): 2-5% by weight
Emulsifier (e.g., Lecithin): 0.5-2% by weight
Stabilizer (e.g., Xanthan Gum): 0.1-1% by weight

### Optional Ingredients:

Natural essential oils may be added for their therapeutic properties and to provide a pleasant scent. These should be used sparingly to prevent potential skin irritation.

In an exemplary embodiment, the ratios of the essential oils are as follows:
Essential Oils (e.g., Lavender, Chamomile): 0.1-1% by weight.

The above ratios and ingredients provide a guideline for the preferred embodiment of the invention. However, variations in the ratios and substitutions of similar ingredients are possible and may be made to tailor the product to specific needs or to comply with regulatory requirements in different jurisdictions. The detailed description and exemplary ratios provided herein enable one skilled in the art to produce the invention without undue experimentation.

### Preparation

The preparation of the preferred embodiment involves the careful mixing of the mushroom extracts with the aloe vera gel to create a base mixture. The non-comedogenic carrier oils, along with any additional emollients, emulsifiers, and stabilizers, are then blended into the base mixture until a homogenous consistency is achieved. The process is conducted under sanitary conditions to prevent contamination and ensure the product's purity and safety.

In the preferred embodiment, the preparation involves a step-by-step process where the mushroom extracts are first blended with the aloe vera gel to create a uniform base. The carrier oils, waxes, and butters are gently heated and then slowly combined with the base mixture. The vitamins, emollients, emulsifiers, and stabilizers are added sequentially under constant mixing to ensure homogeneity. If essential oils are used, they are incorporated last, after the mixture has cooled to an appropriate temperature to preserve their integrity.

While the preferred embodiment of the invention is a cream, the formulation is versatile and can be adapted into other forms such as lotions, gels, or balms to suit different preferences and application needs. This adaptability demonstrates the invention's broad applicability and potential for use in a variety of skin care contexts beyond tattoo aftercare, including but not limited to the treatment of minor skin abrasions, burns, and other forms of dermatological trauma.

The manufacturing process for the preferred embodiment is designed to be compatible with standard cosmetic production equipment, ensuring that it can be easily and economically scaled to meet market demand. Quality control measures are in place at every stage of production, from raw material sourcing to final packaging, to ensure that each batch of the product meets the high standards set forth in the patent. These measures are critical for maintaining the consistency, quality, and integrity of the product as it is produced and distributed.

### Application

The aftercare product is intended for external use only and should be applied generously to both fresh and aging tattoos. Application should begin approximately 48-72 hours after the tattooing process, once the dressing is removed, and continue as per the artist's aftercare instructions or until the skin is fully healed. The product's composition is safe and suitable for all skin types, being inert, non-toxic, and hypoallergenic, with a very low probability of causing adverse or allergic reactions.

The final product should be applied to the tattooed area in a thin layer, ensuring full coverage. It can be used 2-3 times daily or as needed to maintain hydration and promote healing. The product should be stored in a cool, dry place and used within the shelf-life specified by the stability testing.

For ongoing maintenance, the product can be used to keep the tattooed skin moisturized and protected from UV radiation. The frequency of application for long-term care can be adjusted based on individual needs and environmental factors.

### Modifications and Variations

While the preferred embodiment of the invention has been described, it is to be understood that variations and modifications can be made without departing from the spirit and scope of the invention. The proportions of active and inactive ingredients may vary depending on the desired properties of the final product, such as consistency, absorption rate, and shelf-life. The invention is adaptable to include additional natural extracts or compounds that may enhance its healing and protective qualities.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

I claim:

1. A topical aftercare composition for tattooed skin, comprising:
a mixture of medicinal mushroom extracts;
aloe vera gel;
non-comedogenic carrier oils;
wherein the composition is formulated to promote healing of tattooed skin and provide UV radiation protection.

2. The topical aftercare composition of claim 1, wherein the medicinal mushroom extracts include Chaga, Lion's Mane, and Reishi.

3. The topical aftercare composition of claim 2, wherein the Chaga Extract is present in a weight percentage of 5-10%, the Lion's Mane Extract is present in a weight percentage of 3-7%, and the Reishi Extract is present in a weight percentage of 3-7%.

4. The topical aftercare composition of claim 1, wherein the non-comedogenic carrier oils include grapeseed oil and tea tree oil.

5. The topical aftercare composition of claim 4, wherein the grapeseed oil is present in a weight percentage of 5-10% and the tea tree oil is present in a weight percentage of 0.5-2%.

6. The topical aftercare composition of claim 1, further comprising natural waxes and butters.

7. The topical aftercare composition of claim 6, wherein the natural waxes and butters include beeswax and shea butter, with beeswax present in a weight percentage of 1-5% and shea butter present in a weight percentage of 2-8%.

8. The topical aftercare composition of claim 1, further comprising vitamins.

9. The topical aftercare composition of claim 8, wherein the vitamins include Vitamin A and Vitamin C, with Vitamin A present in a weight percentage of 0.1-0.5% and Vitamin C present in a weight percentage of 0.1-0.5%.

10. The topical aftercare composition of claim 1, further comprising emollients, emulsifiers, and stabilizers.

11. The topical aftercare composition of claim 10, wherein the emollients, emulsifiers, and stabilizers include glycerin, lecithin, and xanthan gum, with glycerin present in a weight percentage of 2-5%, lecithin present in a weight percentage of 0.5-2%, and xanthan gum present in a weight percentage of 0.1-1%.

12. The topical aftercare composition of claim 1, further comprising essential oils.

13. The topical aftercare composition of claim 12, wherein the essential oils include lavender and chamomile, with the essential oils present in a weight percentage of 0.1-1%.

14. The topical aftercare composition of claim 1, wherein the composition is formulated as a cream, lotion, gel, or balm.

15. The topical aftercare composition of claim 1, wherein the composition is formulated to be applied to tattooed skin approximately 48-72 hours after tattooing and to be used for ongoing maintenance of the tattooed skin.

16. The topical aftercare composition of claim 1, wherein the composition is hypoallergenic and non-irritating, suitable for all skin types.

17. The topical aftercare composition of claim 1, wherein the composition is stable under a range of temperatures and resistant to photodegradation.

18. The topical aftercare composition of claim 1, wherein the composition is manufactured using a proprietary extraction process for the medicinal mushroom extracts and a novel emulsification technique for combining oil and water-based components.

19. The topical aftercare composition of claim 1, wherein the composition includes a controlled-release mechanism for sustained delivery of the active ingredients.

20. The topical aftercare composition of claim 1, wherein the composition is produced and distributed with quality control measures ensuring consistency, quality, and integrity of the product.
